(19) United States
(12) Patent Application Publication
(10) Pub. No.: US 2014/0316380 A1
(43) Pub. Date: Oct. 23, 2014

(54) DEVICES AND METHODS FOR MAINTAINING AN ASEPTIC CATHETER ENVIRONMENT

(71) Applicant: Nicholas B. Davis, Shelbyville, IN (US)

(72) Inventor: Nicholas B. Davis, Shelbyville, IN (US)

(21) Appl. No.: 14/257,279

(22) Filed: Apr. 21, 2014

Related U.S. Application Data
(60) Provisional application No. 61/862,874, filed on Aug. 6, 2013.
(60) Provisional application No. 61/814,198, filed on Apr. 19, 2013.

(51) Int. Cl. A61M 1/36; A61M 25/00

(52) U.S. Cl. CPC A61M 1/3653; A61M 25/0017; A61M 2025/0019
USPC 604/508; 604/523; 604/535

(57) ABSTRACT

A device for maintaining an aseptic catheter environment is provided. The device includes a first housing and a second housing. The first housing and the second housing are coupled to one another by way of at least one coupler. The first housing and the second housing are configured to engage one another when in a closed configuration and to define an interior space when in the closed configuration. At least one aperture and/or notch is defined within at least one of the first housing and the second housing. The at least one aperture and/or notch is sized and shaped to allow at least part of a catheter to be positioned therethrough.

### CROSS-REFERENCE TO RELATED APPLICATIONS

The present application claims priority to U.S. Provisional Patent Application No. 61/814,198, having a filing date of Apr. 19, 2013 and a title of “ANTISEPTIC CATHETER CAP”, and U.S. Provisional Patent Application No. 61/862,874, having a filing date of Aug. 6, 2013 and a title of “DEVICES AND METHODS FOR MAINTAINING AN ASEPTIC CATHETER ENVIRONMENT”, both of which are herein incorporated in their entirety.

### BACKGROUND

Infections stemming from unsanitary bodily catheters, including those used for dialysis, can both be costly and painful. Currently, when a patient or technician prepares his or her implanted bodily catheters for dialysis or another procedure, the external surfaces of the catheters must be cleaned prior to connection to dialysis or other machines. Improper or ineffective cleaning can lead to “dirty” connections, which often lead to costly, painful, life-threatening infections. Over five million central catheters are placed annually, resulting in 250,000 bloodstream infections. In the case of urinary catheters, such catheters result in 500,000 annual urinary tract infections.

Devices and methods useful to reduce or prevent bacterial or other infection in connection with various bodily catheters would solve this problem and be well received in the marketplace.

### BRIEF SUMMARY

The disclosure of the present application addresses the reduction and/or prevention of bacterial or other infection in connection with various bodily catheters, including those used for dialysis, such as central venous catheters as well as peripherally inserted central catheters (PICCs or PICC lines). Such bodily catheters may be used for various types of dialysis procedures, including, but not limited to, hemodialysis, perotineal dialysis, intestinal dialysis, hemodialysis, and other types of dialysis, such as liver dialysis, as well as for various injections and/or suction procedures, including those useful to remove excess fluid from various areas within the body. The devices of the present disclosure, for example, may be used by to maintain an aseptic bodily catheter environment when not engaging in dialysis or when engaging in dialysis, as such devices “clean” the external portions of bodily catheters, dialyzer bloodline connectors, caps, etc., before, during, and after dialysis. Such devices may also provide more freedom of movement to do typical tasks, such as showering, with a reduced risk of infection from the tasks. Such devices may also be relatively thin and lightweight to provide a comfortable experience when in use. In various embodiments, such devices may have rounded portions for additional comfort when the device comes in contact with a body. The subject body may be a human or animal body.

In an exemplary embodiment of a device for maintaining an aseptic catheter environment of the present disclosure, the device comprises a first housing and a second housing, the first and second housings coupled to one another by way of at least one coupler and configured to engage one another when in a closed configuration and to define an interior space when in the closed configuration, and at least one aperture and/or notch defined within the first housing and/or the second housing, the aperture sized and shaped to permit a catheter to be positioned therethrough.

In at least one exemplary embodiment of a device for maintaining an aseptic catheter environment of the present disclosure, the device comprises a first housing and a second housing, the first housing and the second housing coupled to one another by way of at least one coupler, the first housing and the second housing configured to engage one another when in a closed configuration and to define an interior space when in the closed configuration, and at least one aperture and/or notch defined within at least one of the first housing and the second housing, the at least one aperture and/or notch sized and shaped to allow at least part of a catheter to be positioned therethrough. In another embodiment, the at least one coupler comprises a hinge coupled to the first housing and the second housing so that the first housing and the second housing hingedly move about one another. In yet another embodiment, the at least one coupler comprises at least one clip, the at least one clip operable to reversibly secure the first housing to the second housing.

In at least one exemplary embodiment of a device for maintaining an aseptic catheter environment of the present disclosure, at least one of the first housing and the second housing comprises a housing gasket positioned around at least a portion of a perimeter of the first housing and/or the second housing. In an additional embodiment, the first housing comprises the housing gasket, and wherein the second housing comprises a groove positioned around at least a portion of a perimeter of the second housing, the groove configured to receive the housing gasket. In yet an additional embodiment, the housing gasket provides a fluid-tight seal when the first housing and the second housing engage each other in the closed configuration. In another embodiment, the device further comprises at least one aperture gasket positioned at or near the at least one aperture and/or notch, wherein the at least one aperture gasket provides a fluid-tight seal when a catheter is positioned within the aperture and/or notch.

In at least one exemplary embodiment of a device for maintaining an aseptic catheter environment of the present disclosure, the device further comprises one or more saturable materials positioned within the interior space of the device. In an additional embodiment, the one or more saturable materials are selected from the group consisting of a sponge, a fabric, and/or a gauze.

In at least one exemplary embodiment of a device for maintaining an aseptic catheter environment of the present disclosure, the device further comprises a fluid sac containing an antiseptic fluid, the fluid sac positioned upon at least one of the first housing and the second housing. In another embodiment, the fluid sac is capable of being ruptured by way of a rupturer positioned within the device. In various embodiments, the rupturer is selected from the group consisting of a pin, a needle, a portion of the first housing, and a portion of the second housing. In an additional embodiment, and when the fluid sac becomes ruptured, the fluid sac releases fluid within the interior space of the device to create an aseptic environment within the interior space of the device. In yet an additional embodiment, the fluid sac is capable of being ruptured by way of closing the device, whereby pressure from at least one of the first housing and the second housing causes the fluid sac to rupture. In another embodiment, and when the fluid sac becomes ruptured, the fluid sac releases fluid within the interior space of the device to create an aseptic environment within the interior space of the device.

In at least one exemplary embodiment of a device for maintaining an aseptic catheter environment of the present disclosure, the at least one aperture and/or notch comprises at least two apertures and/or notches. In an additional embodiment, the at least one aperture and/or notch comprises at least four apertures and/or notches, wherein at least two of the at least four apertures and/or notches are positioned relatively opposite to at least two other of the at least four apertures and/or notches. In yet an additional embodiment, the device further comprises a strap coupled thereto, the strap configured to be positioned about a portion of a body. In another embodiment, the strap further comprises a strap adjuster to allow adjustment of the strap.

In at least one exemplary embodiment of a device for maintaining an aseptic catheter environment of the present disclosure, the device comprises a first housing and a second housing, the first housing and the second housing coupled to one another by way of at least one hinge, the first housing and the second housing configured to engage one another when in a closed configuration and to define an interior space when in the closed configuration, at least one clip coupled to the first housing or the second housing, the at least one clip operable to reversibly secure the first housing to the second housing, one or more saturable materials positioned within the interior space of the device, a fluid sac containing an antiseptic fluid, the fluid sac positioned upon at least one of the first housing and the second housing, and at least one aperture and/or notch defined within at least one of the first housing and the second housing, the at least one aperture and/or notch sized and shaped to allow at least a portion of a catheter to be positioned therethrough.

In at least one exemplary embodiment of a device for maintaining an aseptic catheter environment of the present disclosure, the device comprises a first housing and a second housing, the first housing and the second housing coupled to one another by way of at least one hinge, the first housing and the second housing configured to engage one another when in a closed configuration and to define an interior space when in the closed configuration, at least one clip coupled to the first housing or the second housing, the at least one clip operable to reversibly secure the first housing to the second housing, one or more saturable materials positioned within the interior space of the device, a fluid sac containing an antiseptic fluid, the fluid sac positioned upon at least one of the first housing and the second housing, and at least four apertures and/or notches defined within at least one of the first housing and the second housing, wherein at least two of the at least four apertures and/or notches are positioned relatively opposite to at least two other of the at least four apertures and/or notches, the at least four apertures and/or notches sized and shaped to allow at least portions of catheters to be positioned therethrough.

In at least one exemplary embodiment of a method of using a device for maintaining an aseptic catheter environment of the present disclosure, the method comprises the steps of placing a distal end of a bodily catheter within a aperture and/or a notch of a device for maintaining an aseptic catheter environment, closing the device, and activating the device to release an antiseptic fluid within the device. In another embodiment, the step of activating the device is performed by applying pressure to the device to cause the antiseptic fluid within the device to be released.

In at least one exemplary embodiment of a method of proceeding with dialysis using a device for maintaining an aseptic catheter environment of the present disclosure, the method comprises the steps of removing a device for maintaining an aseptic catheter environment from one or more bodily catheters positioned at least partially therein, removing caps positioned at or near a distal end of the one or more bodily catheters, connecting the one or more bodily catheters to one or more dialyzer catheters, removing clamps positioned upon the one or more bodily catheters, and proceeding with dialysis.

In at least one exemplary embodiment of a method of proceeding with dialysis using a device for maintaining an aseptic catheter environment of the present disclosure, the method comprises the steps of removing caps positioned at or near the distal ends of bodily catheters, connecting the bodily catheters to dialyzer catheters, positioning a device for maintaining an aseptic catheter environment about at least a portion of the bodily catheters and at least a portion of the dialyzer catheters, closing the device, activating the device to release an antiseptic fluid within the device, removing clamps positioned upon the bodily catheters, and proceeding with dialysis.

In at least one exemplary embodiment of an adhesive device of the present disclosure, the device comprises a pouch sized and shaped to permit a portion of a bodily catheter extending from a body to be positioned therein, and an adhesive ring coupled to the pouch, wherein when the bodily catheter is positioned within the pouch, the adhesive ring is capable of adhering to the body to form at least a substantially fluid and/or air tight seal.

### BRIEF DESCRIPTION OF THE DRAWINGS

FIG. 1A shows a front view of an exemplary embodiment of a device for maintaining an aseptic catheter environment according to the present disclosure;

FIGS. 1B and 1C show side views of an exemplary device according to an embodiment of the present disclosure;

FIG. 1D shows a top view of an exemplary device according to an embodiment of the present disclosure;

FIGS. 2A and 2B show views of open devices for maintaining an aseptic catheter environment according to embodiments of the present disclosure;

FIGS. 2C and 2D show cross-sectional views of a device for maintaining an aseptic catheter environment according to embodiments of the present disclosure;

FIG. 2E shows steps of an exemplary method for using an exemplary device for maintaining an aseptic catheter environment according to an embodiment of the present disclosure;

FIG. 2F shows a view of an open device for maintaining an aseptic catheter environment with bodily catheters positioned therein according to an embodiment of the present disclosure;

FIG. 2G shows a view of a closed device for maintaining an aseptic catheter environment with bodily catheters positioned therein according to an embodiment of the present disclosure;

FIG. 2H shows steps of an exemplary method for proceeding with dialysis after using an exemplary device for maintaining an aseptic catheter environment according to an embodiment of the present disclosure;

FIG. 3A shows a front view of an exemplary embodiment of device for maintaining an aseptic catheter environment according to an embodiment of the present disclosure;

FIGS. 3B and 3C show side views of an exemplary device according to an embodiment of the present disclosure;

FIG. 3D shows a top view of an exemplary device according to an embodiment of the present disclosure;

FIG. 3E shows a bottom view of an exemplary device according to an embodiment of the present disclosure;

FIG. 4 shows a view of an open device for maintaining an aseptic catheter environment with bodily catheters and dialyzer catheters positioned therein according to an embodiment of the present disclosure;

FIG. 5 shows steps of an exemplary method for using an exemplary device for maintaining an aseptic catheter environment according to an embodiment of the present disclosure;

FIG. 6 shows a front view of an exemplary embodiment of a device for maintaining an aseptic catheter environment with a strap coupled thereto according to an embodiment of the present disclosure;

FIG. 7 shows an exemplary adhesive device according to an embodiment of the present disclosure; and

FIG. 8 shows an exemplary adhesive device with a bodily catheter positioned therein according to an embodiment of the present disclosure.

FIG. 9 shows a perspective view of a device for maintaining an aseptic catheter environment according to at least one embodiment of the present disclosure;

FIG. 10 shows a plan view of a top housing of a device for maintaining an aseptic catheter environment according to at least one embodiment of the present disclosure;

FIG. 11 shows a plan view of a bottom housing of a device for maintaining an aseptic catheter environment according to at least one embodiment of the present disclosure;

FIG. 12 shows a perspective view of a top seal of a device for maintaining an aseptic catheter environment according to at least one embodiment of the present disclosure;

FIG. 13 shows a perspective view of a bottom seal of a device for maintaining an aseptic catheter environment according to at least one embodiment of the present disclosure;

FIG. 14 shows a perspective view of a box assembly of a device for maintaining an aseptic catheter environment according to at least one embodiment of the present disclosure;

FIG. 15 shows a perspective view of a device for maintaining an aseptic catheter environment according to at least one embodiment of the present disclosure;

FIG. 16 shows a perspective view of seal of a device for maintaining an aseptic catheter environment according to at least one embodiment of the present disclosure;

FIG. 17 shows a perspective view of seal of a device for maintaining an aseptic catheter environment according to at least one embodiment of the present disclosure;

FIG. 18 shows a perspective view of a device for maintaining an aseptic catheter environment according to at least one embodiment of the present disclosure;

FIG. 19 shows a perspective view of a device for maintaining an aseptic catheter environment according to at least one embodiment of the present disclosure;

FIG. 20 shows a perspective view of a device for maintaining an aseptic catheter environment according to at least one embodiment of the present disclosure; and

FIG. 21 shows a perspective view of plug of a device for maintaining an aseptic catheter environment according to at least one embodiment of the present disclosure.

### DETAILED DESCRIPTION

The disclosure of the present application provides various devices and methods for maintaining an aseptic catheter environment. For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended. Further, where the embodiments illustrated in the drawings, and specific language will be used to describe the same, refer a body or a patient's body, it will be understood that the subject body may be a human body or an animal body, as the use of catheters in vivo is known in both human and veterinary medicine.

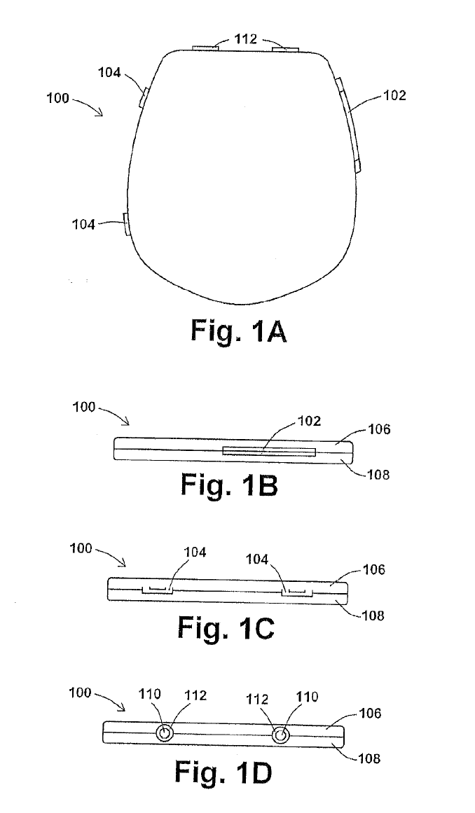

An exemplary embodiment of a device for maintaining an aseptic catheter environment of the present disclosure is shown in FIGS. 1A-1D. As shown in the front view shown in FIG. 1A, an exemplary embodiment of device 100 comprises a hinge 102 and one or more clips 104. Hinge 102 and clips 104, in at least one embodiment, are coupled to device 100 opposite one another to facilitate the opening, closing, and securing of a first housing 106 and a second housing 108 to one another as shown in FIGS. 1B-1D. As shown in FIGS. 1A-1D, device 100 is in a “closed” configuration, whereby first housing 106 and second housing 108 define a volume within device 100.

First housing 106, second housing 108, hinge 102, and clips 104 may comprise any number of materials known in the art suitable for an exemplary device 100 as referenced herein including, but not limited to, various plastics and/or metals. In at least one embodiment, first housing 106, second housing 108, hinge 102, and clips 104 each comprise polypropylene plastic. The materials, in at least one embodiment, should be of sufficient rigidity so that the various embodiments of devices 100 of the present disclosure may sufficiently operate, for example, to remain closed when bodily catheters are positioned within device 100, and to form an appropriate seal when device 100 is in a “closed” configuration. In at least one embodiment, first housing 106, second housing 108, and hinge 102 are unitary in nature by way of being formed as one molded plastic piece, for example.

FIG. 1B shows a side view of an exemplary device 100 of the present disclosure. As shown in FIG. 1B, device 100 comprises a first housing 106 and a second housing 108, whereby first housing 106 and second housing 108 are coupled to one another by way of hinge 102. Hinge 102 may comprise any number of hinge mechanisms known in the art, including, but not limited to, a pliable plastic hinge, so that hinge 102 permits first housing 106 and second housing 108 to hingedly move in relation to one another.

FIG. 1C shows a side view of an exemplary device 100 of the present disclosure. As shown in FIG. 1C, device 100 comprises a first housing 106 and a second housing 108, whereby first housing 106 and second housing 108 may be reversibly secured to one another by way of one or more clips 104. Clips 104, as shown in FIG. 1C, may comprise clips extending from either the first housing 106 and the second housing 108, and operate to reversibly secure the first housing 106 to the second housing 108. For purposes of the present disclosure, hinge 102 and clips 104 may be generally referred to as a “coupler” or “couplers.” In such an embodiment, for example, clips 104, and not a hinge 102, is used to couple first housing 106 to second housing 108.

A top view of an exemplary embodiment of a device 100 of the present disclosure is shown in FIG. 1D. As shown in FIG. 1D, device 100 defines at least one aperture 110 positioned therethrough. In the exemplary embodiment shown in FIG. 1D, apertures 110 are partly defined by first housing 106 and partly defined by second housing 108. In another embodiment, apertures 110 may be fully defined within either first housing 106 and/or second housing 108. In addition, and as shown in FIGS. 1A and 1D, device 100 may further comprise one or more aperture gaskets 112 positioned at or near apertures 110, whereby aperture gaskets 112 facilitate a fluid-tight seal at the site of aperture(s) 110 when, for example, a catheter (not shown) is positioned therethrough as discussed hereinafter.

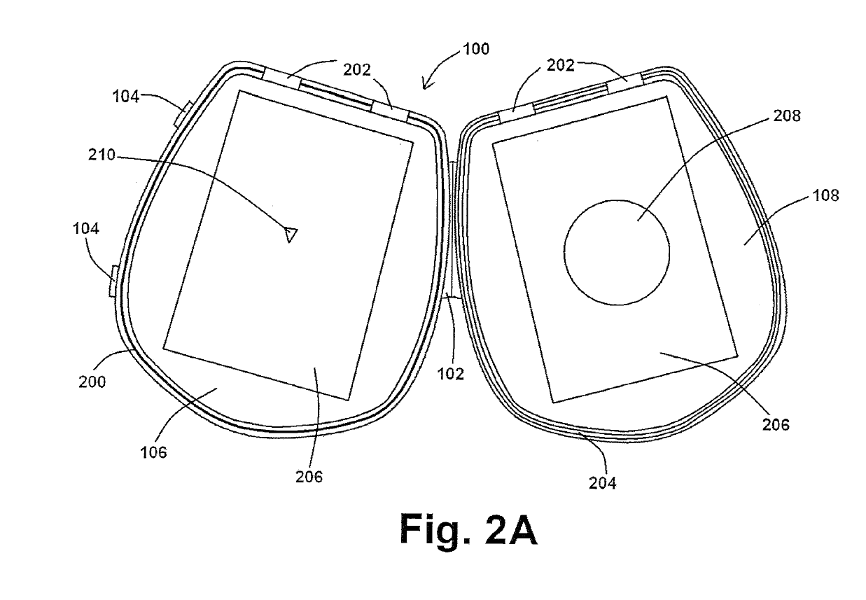

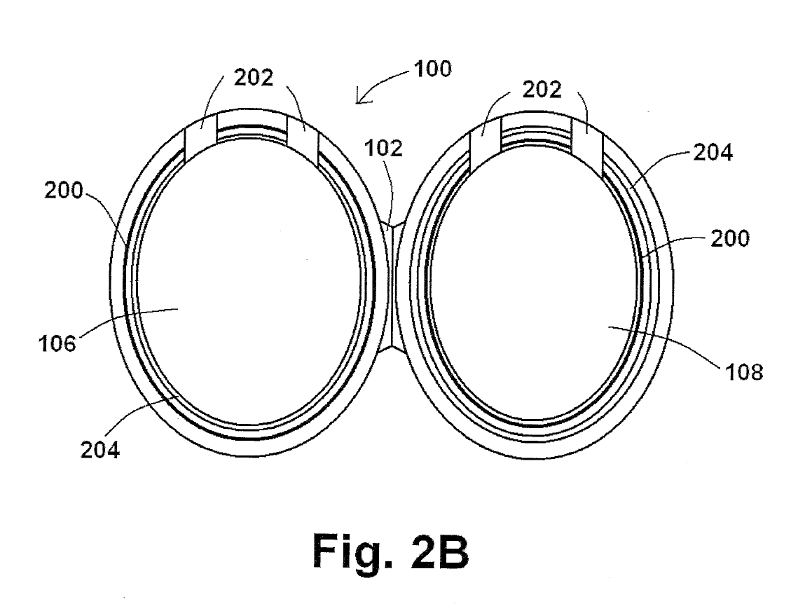

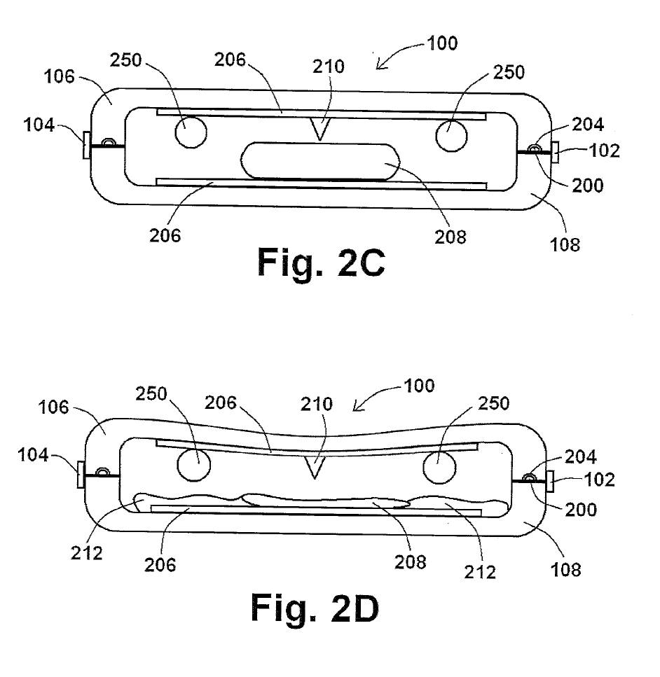

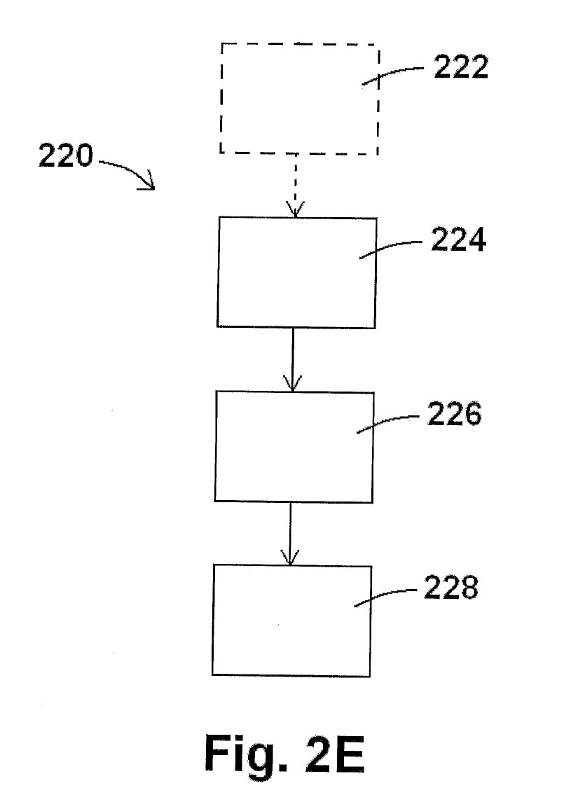

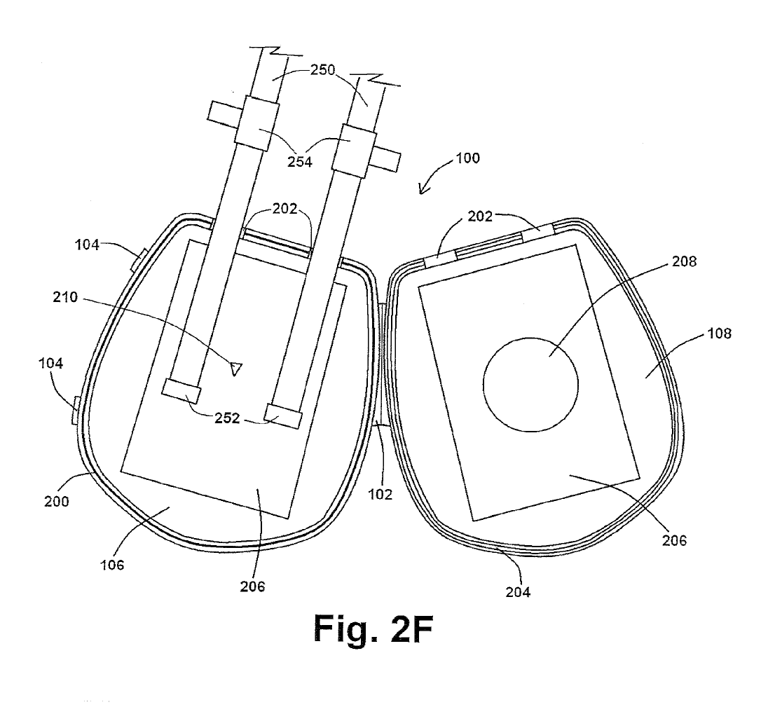

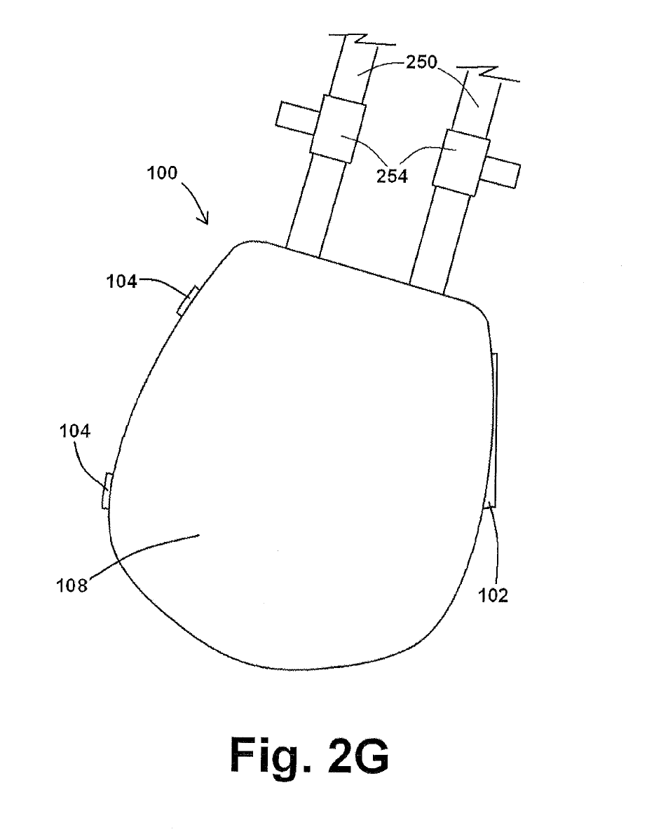

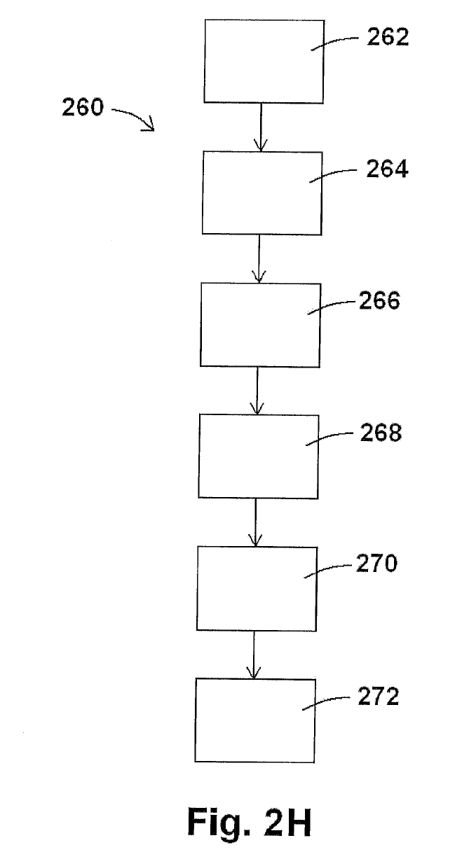

An open view of an exemplary embodiment of a device 100 of the present disclosure is shown in FIG. 2A. As shown in FIG. 2A, device 100 may comprise a first housing 106 coupled to a second housing 108 by way of hinge 102, whereby clips 104 positioned along first housing 106 may operate to engage second housing 108 when device 100 is in a “closed” configuration. Alternative embodiments, as discussed herein, may appear similar to the exemplary embodiment of device 100 shown in FIG. 2A but may include, for example, additional clips 104 in lieu of hinge 102, so that clips 104 may reversibly secure first housing 106 to second housing 108. Furthermore, clips 104 may appear on either or both first housing 106 and/or second housing 108.

As shown in FIG. 2A, device 100 may further comprise an optional housing gasket 200 positioned around at least part of the perimeter of first housing 106 and/or second housing 108. Housing gasket 200, when device 100 is in a “closed” configuration, facilitates a fluid-tight or substantially fluid-tight seal so that fluid positioned within device 100 will not leak outside device 100. Such a fluid tight seal may exist in connection with a fluid tight seal present by way of catheters being positioned within apertures 110, shown in FIG. 2A as being defined by notches 202 positioned within first housing 106 and second housing 108, so that when device 100 is in a “closed” configuration, apertures 110 are defined by way of notches 202. Aperture gaskets 112 (shown in FIG. 1D), and in an exemplary embodiment, may be positioned at or near notches 202 (as shown in FIG. 2A) to further facilitate a fluid-tight seal when, for example, a catheter is positioned therethrough and in contact therewith.

In addition to the foregoing, one or more grooves 204 may be positioned in a housing opposite the housing comprising housing gasket 200, whereby housing gasket 200 may engage groove 204 when the device is in a “closed” configuration. In at least one embodiment, and as shown in FIG. 2B, each housing comprises a housing gasket 200 and a groove 204, so that the housing gasket 200 of first housing 106 engages groove 204 of second housing 108, and housing gasket 200 of second housing 108 engages groove 204 of first housing 106, forming an fluid and/or air tight seal.

In the embodiment shown in FIG. 2A, device 100 further comprises one or more saturable materials 206 positioned “inside” of device 100. FIG. 2A shows saturable materials 206 positioned upon first housing 106 and second housing 108, but any number of saturable materials 206 may be used, including one saturable material 206. Saturable materials 206 may comprise any number of materials that can become either partially or fully saturated by a fluid, such as, for example, a sponge, a fabric, or a gauze, and may either be initially provided “dry” (i.e., without any fluid) or “wet” (i.e., with, saline, an antiseptic fluid, etc., positioned thereon).

Such saturable materials 206 may become either partially or fully saturated by a fluid initially positioned within, for example, fluid sac 208 as shown in FIG. 2A. Fluid sac 208, in at least one embodiment, may be ruptured by way of one or more rupturers 210, which may comprise, for example, a pin or a needle coupled to first housing 106 and/or second housing 108. In alternative embodiments, rupturer 210 may comprise a relatively “sharp” portion protruding from first housing 106 and/or second housing 108, or may comprise a portion of first housing 106 and/or second housing 108 which is not relatively “sharp” but is otherwise flexible/pliable so that when a user of device 100 exerts pressure on rupturer 210, rupturer 210 facilitates the rupture of fluid sac 208. As shown in the exemplary embodiment of device 100 shown in FIG. 2A, rupturer 210 is coupled to, or formed as a part of, first housing 106. In at least one embodiment, rupturer 210 is positioned within device 100 at a location so not to potentially puncture any bodily catheter positioned therein. Such puncture prevention may be accomplished in several ways including, but not limited to, device 100 design elements, grooves within saturable elements 206 to receive the catheters, and/or physical barriers within device 100 to prevent movement of the catheters within device 100.

In at least one embodiment of a device 100 of the present disclosure, the closure of the device 100 itself causes fluid sac 208 to rupture. For example, and in at least one embodiment, fluid sac 208 is sized and shaped and contains enough fluid so that when device 100 is closed (with our without portions of catheters therein), the closure causes pressure to be exerted upon fluid sac 208 causing fluid sac to rupture.

Fluid within fluid sac 208 may comprise, for example, any number of antiseptic fluids including, but not limited to, betadine, iodine, various alcohols, various chlorines and/or hypochlorites, chlorhexidine, hypoallergenic solutions, and/or any other antibacterial agents known in the art. Such fluids, when released from fluid sac 208, can enter an interior space of device 100 (created when device 100 is in a “closed” configuration), and can serve as an antiseptic/antibacterial agent and effectively create an “aseptic” environment within device 100.

FIGS. 2C and 2D show a cross-sectional view of an exemplary device 100 of the present disclosure. As shown in FIG. 2C, device 100 comprises a rupturer 210 coupled/affixed to first housing 106, positioned relative to a fluid sac 208 present within the space between first housing 106 and second housing 108. When device 100 is “closed,” as shown in FIG. 2C, housing gasket 200 is positioned within groove 204, forming a fluid and/or air tight seal (or substantially a fluid and/or air tight seal). Squeezing first housing 106, for example and as shown by way of the deflection of first housing 106 shown in FIG. 2D, causes rupturer 210 to rupture fluid sac 208, thereby releasing fluid 212 from fluid sac 208 into the volume/space within the closed device 100. Fluid 212 may then be absorbed by saturable material(s) 206 present within device 100 and also coat some or all of bodily catheters 250 positioned within device 100, creating an aseptic environment as referenced herein. Such an embodiment of device 100 shown in FIGS. 2C and 2D may be referred to as a “single-use device,” as fluid sac 208 is ruptured to release fluid 212 within device 100.

An exemplary method of using a device 100 of the present application is shown in FIG. 2E and described below. For example, a device 100 as shown in FIGS. 1A-2A may be used by a dialysis patient having one or more catheters extending from the patient's body which are in communication with the patients circulatory system (e.g., a patient's jugular vein, subclavian vein, vena cava, etc.). Such catheters may have single or multiple lumens. In the exemplary embodiment of device 100 shown in FIGS. 1A-2A, the patient has one bodily catheter (with a double lumen) that branches into two catheters (one for blood/fluid flow from the patient's body and one for blood/fluid flow back into the patient's body to the dialyzer after the blood/fluid is processed through a dialyzer), or the patient may have two bodily catheters extending from his or her body serving a similar purpose as described herein.

In an exemplary method 220 of the present disclosure, and as shown in FIG. 2E, prior to a new dialysis session, or sometime after a prior dialysis session, a patient may optionally “clean” his or her bodily catheters by any number of means known in the art (optional cleaning step 222) to remove and/or neutralize, for example, excess bacteria around the bodily catheters. Regardless of prior catheter cleaning, the patient, or a user of device 100, may then place device 100 about his or her bodily catheters by placing the distal ends of the catheters within apertures 110 or notches 202 of device 100 so that the exposed ends of the catheters are positioned within device 100 (catheter placement step 224). After the bodily catheters are positioned within device 100, device 100 may be “closed” about the distal ends of the bodily catheters (closing step 226). After device 100 is “closed,” the patient, and/or a user of device 100, may “activate” device 100 by, for example, squeezing device 100 so that rupturer 210 may rupture fluid sac 208 to release antiseptic fluid within device 100 (activation step 228), creating an aseptic environment. After the antiseptic fluid present within device 100 has been in contact with the distal ends of the bodily catheters, the patient shall be ready to proceed with dialysis without the need to perform any additional/extensive bodily catheter cleaning procedures as the bodily catheters will already be “clean,” thus reducing the likelihood/risk of bacterial or other infection common when connecting the bodily dialysis catheters to a dialyzer. Removal of device 100, in at least one embodiment, shall comprise the steps of “opening” device 100 and removing device 100 from the bodily catheters, thereby allowing the patient to connect the bodily catheters to a dialyzer to proceed with dialysis.

An embodiment of device 100 of the present disclosure showing bodily catheters 250 positioned therein is shown in FIG. 2F. As shown in FIG. 2F, the distal ends of bodily catheters 250 may be positioned within notches 202 (or apertures 110) of device 100, with the bodily catheters 250 being capped using, for example, caps 252, and optionally clamped using, for example, clamps 254. Caps 252 and/or clamps 254 may function to prevent the flow of fluid (blood in the case of hemodialysis) from a patient's body out of bodily catheters 250. Upon “closing” and “activating” device 100 as described above, the distal ends of bodily catheters 250, including caps 252, shall be within an aseptic environment created by device 100, thus reducing the risk of potential infection when the patient proceeds with a dialysis procedure as described below. Device 100, shown in an “open” configuration in FIG. 2F, may be “closed” as shown in FIG. 2G, whereby bodily catheters 250 are positioned within the interior space/volume of device 100.

In an exemplary method 260 of proceeding with dialysis of the present disclosure, and as shown in FIG. 2G, a patient, or a user of device 100, may remove device 100 from bodily catheters 250 (removal step 262) and prepare a dialyzer for dialysis (dialyzer preparation step 264, which may include any number of tasks known in connection with performing dialysis) prior to proceeding with dialysis. Removal step 262 and dialyzer preparation step 264 may be performed in either order, but are shown in the exemplary method shown in FIG. 2G as having removal step 262 precede dialyzer preparation step 264. The patient, or a user of device 100, may then remove caps 252 (leaving clamps 254 in place to prevent fluid flow from the patient's body) (cap removal step 266), and then connect bodily catheters 250 to the corresponding dialzyer catheters/connectors (connection step 268). The patient, or a user of device 100, may then remove clamps 254 (clamp removal step 270) and proceed with dialysis (dialysis step 272).

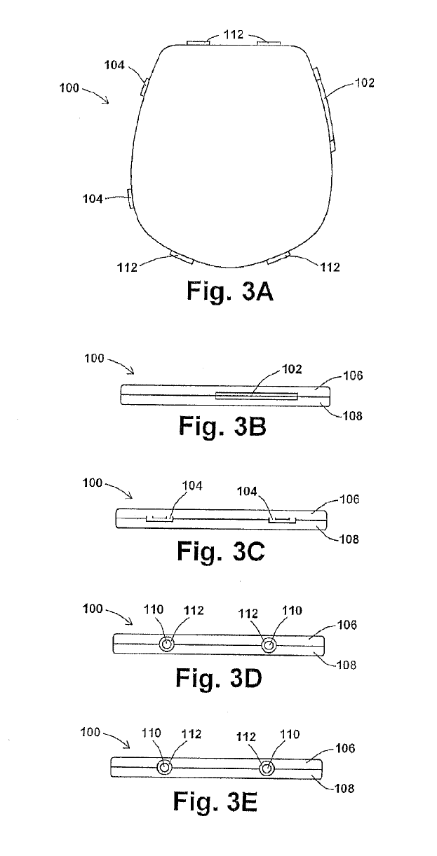

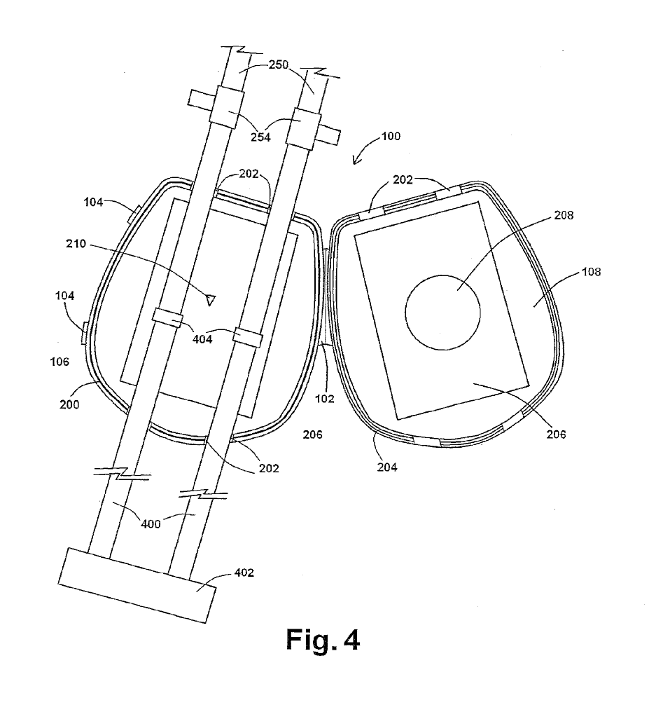

Another exemplary embodiment of a device for maintaining an aseptic catheter environment of the present disclosure is shown in FIGS. 3A-3E. As shown in the front view shown in FIG. 3A, an exemplary embodiment of device 100 may comprise a hinge 102 and one or more clips 104. Hinge 102 and clips 104, in at least one embodiment, are coupled to device 100 opposite one another to facilitate the opening, closing, and securing of a first housing 106 and a second housing 108 to one another as shown in FIGS. 3B-3E and 4. First housing 106, second housing 108, hinge 102, and clips 104 may comprise any number of materials known in the art suitable for an exemplary device 100 as referenced herein including, but not limited to, various plastics and/or metals. In at least one embodiment, first housing 106, second housing 108, hinge 102, and clips 104 each comprise polypropylene plastic.

FIG. 3B shows a side view of an exemplary device 100 of the present disclosure. As shown in FIG. 3B, device 100 may comprise a first housing 106 and a second housing 108, whereby first housing 106 and second housing 108 are coupled to one another by way of hinge 102. Hinge 102 may comprise any number of hinge mechanisms known in the art, including, but not limited to, a pliable plastic hinge, so that hinge 102 permits first housing 106 and second housing 108 to hingedly move about one another.

FIG. 3C shows a side view of an exemplary device 100 of the present disclosure. As shown in FIG. 3C, device 100 may comprise a first housing 106 and a second housing 108, whereby first housing 106 and second housing 108 may be reversibly secured to one another by way of one or more clips 104. Clips 104, as shown in FIG. 3C, may comprise clips extending from either the first housing 106 and the second housing 108, and operate to reversibly secure the first housing 106 to the second housing 108. For purposes of the present disclosure, hinge 102 and clips 104 may be generally referred to as a “coupler” or “couplers.”

A top view of an exemplary embodiment of a device 100 of the present disclosure is shown in FIG. 3D, and a bottom view of an exemplary embodiment of a device 100 of the present disclosure is shown in FIG. 3E. As shown in FIGS. 3D and 3E, device 100 defines four apertures 110 positioned therethrough (two apertures 110 at the top and two apertures at the bottom). In an embodiment whereby the bodily catheters 250 or the dialyzer catheters comprise a multi lumen catheter, an exemplary embodiment of device 100 may comprise one aperture positioned at the top of device 100 and one aperture positioned at the bottom of device 100. The number of apertures 110, and/or the physical placement of apertures about device 100, may vary from embodiment to embodiment. In the exemplary embodiments shown in FIGS. 3D and 3E, apertures 110 are partly defined by first housing 106 and partly defined by second housing 108. In other embodiments, apertures 110 may be fully defined within either first housing 106 and/or second housing 108. In addition, and as shown in FIGS. 3A, 3D, and 3E, device 100 may further comprise one or more aperture gaskets 112 positioned at or near apertures 110, whereby aperture gaskets 112 facilitate the presence of a “fluid-tight” seal at the site of aperture(s) 110 when, for example, a catheter (not shown) is positioned therethrough.

An embodiment of device 100 of the present disclosure showing bodily catheters 250 and dialyzer catheters 400 positioned therein is shown in FIG. 4. As shown in FIG. 4, the distal ends of bodily catheters 250 may be positioned within notches 202 (or apertures 110) of device 100, with the bodily catheters 250 being clamped using, for example, clamps 254. Furthermore, the distal ends of dialyzer catheters 400 of dialyzer 402 may be connected to the distal ends of bodily catheters 250 using, for example, connector 404 as shown in FIG. 4, so that the internal lumens of the bodily catheters 250 and the dialyzer catheters 400 are in fluid communication with one another. Upon “closing” and “activating” device 100 as described above, the distal ends of bodily catheters 250 and dialyzer catheters 400, including connector 404 if used to connect bodily catheters 250 to dialyzer catheters 400, shall be within an aseptic environment created by device 100, thus reducing the risk of potential infection when the patient proceeds with a dialysis procedure as described below.

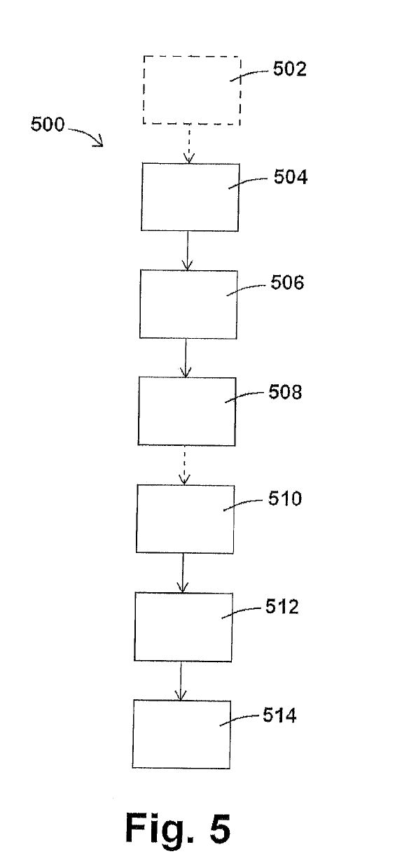

In an exemplary method 500 of proceeding with dialysis of the present disclosure, and as shown in FIG. 5, a patient or technician may optionally “clean” his or her bodily catheters by any number of means known in the art (optional cleaning step 502) to remove and/or neutralize, for example, excess bacteria around the bodily catheters. Regardless of prior catheter cleaning, the patient or technician may remove caps 252 from bodily catheters 250 (leaving clamps 254 in place to prevent fluid flow from the patient's body) (cap removal step 504), and then connect bodily catheters 250 to the corresponding dialzyer catheters 400 (connection step 506). After the bodily catheters 250 are connected to the dialyzer catheters 400 and are positioned within device 100, device 100 may be “closed” about the distal ends of the bodily catheters (closing step 508). After device 100 is “closed,” the patient may “activate” device 100 by, for example, pressing device 100 so that rupturer 210 may rupture fluid sac 208 to release antiseptic fluid within device 100 (activation step 510), creating an aseptic environment. After a desired amount of time has lapsed (so that the antiseptic fluid present within device 100 has been in contact with the distal ends of the bodily catheters 250 and the distal ends of dialyzer catheters 400), the patient shall be ready to proceed with dialysis without the need to perform any additional/extensive bodily catheter 250 and/or dialyzer catheter 400 cleaning procedures those catheters will already be “clean,” thus reducing the likelihood/risk of bacterial or other infection common when performing dialysis. The patient or technician may then remove clamps 254 (clamp removal step 512) and proceed with dialysis (dialysis step 514). Upon completion of dialysis, the patient or technician may remove device 100 (using the reverse of one or more method 500 steps herein), and optionally affix an embodiment of device 100 to his or her bodily catheters (as shown in FIG. 1A) to maintain an aseptic environment when not engaging in dialysis and/or any other medical procedure.

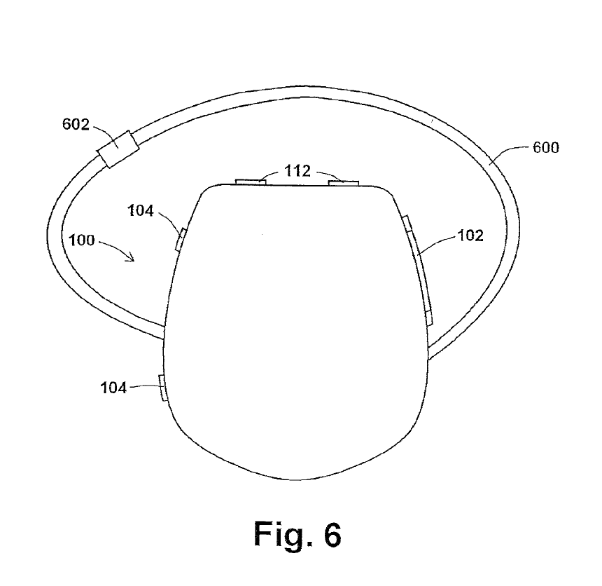

An exemplary embodiment of a device 100 of the present disclosure may be used with one or more straps 600 coupled thereto as shown in FIG. 6. As shown in FIG. 6, device 100 comprises strap 600 coupled thereto, whereby strap 600 is configured to be positioned around a portion of a patient's body, such as the patient's torso. Strap 600 may comprise any number of straps known in the art, including elastic straps, and may be padded for comfort. An exemplary embodiment of a strap 600 may also comprise a strap adjuster 602 to allow the patient to adjust strap 600 as desired. In lieu of the use of strap 600, or in connection with the use, devices 100 may also be removably adhered to a patient's body using one or more adhesives known in the art. Various exemplary embodiments of devices 100 of the present disclosure may incorporate a strap 600 as desired by a user.

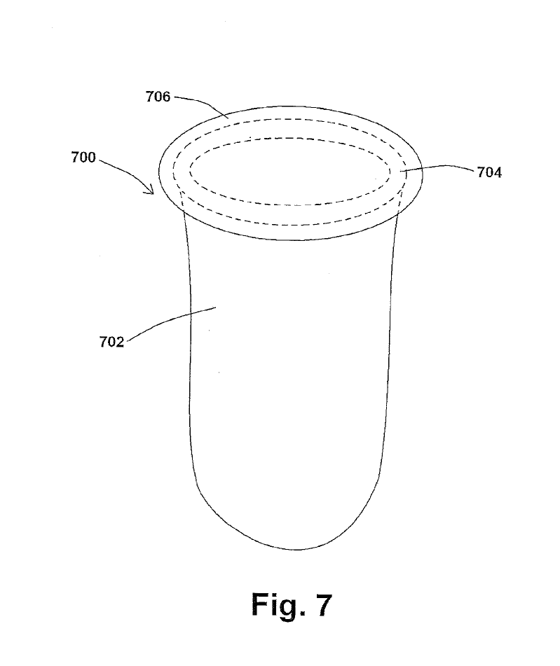

An exemplary embodiment of an adhesive device 700 of the present disclosure is shown in FIG. 7. As shown in FIG. 7, adhesive device comprises a pouch 702 and an adhesive ring 704 coupled to pouch 702. A patient may insert the distal ends of one or more bodily catheters 250 into the pouch 702, and by applying pressure to adhesive ring 704 about a patient's body, adhesive ring 704 may adhere the patient's body to provide an environment within pouch 702 that either reduces or prevents the introduction of fluids, particles, and/or air from entering pouch 702. Adhesive ring 704 may be covered by removable cover 706, such that removal of removable cover 706 exposes an adhesive positioned about adhesive ring 704.

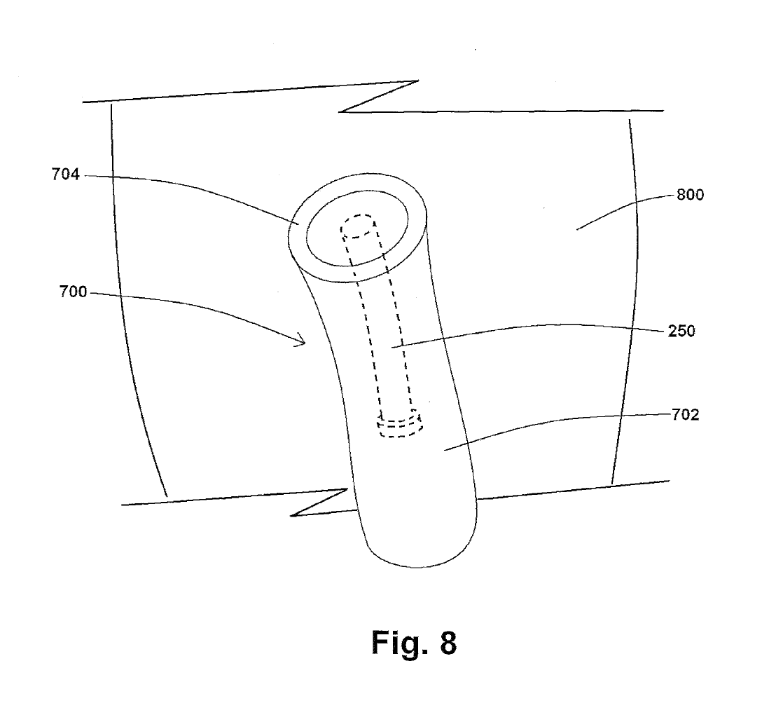

Such an adhesive device 700 may be useful, for example, when a patient wishes to take a shower but not introduce bacteria or other infectious agents to his or her bodily catheters. For example, and as shown in FIG. 8, a bodily catheter 250 may be positioned within device 700, and adhesive ring 704 may be pressed onto a patient's body 800 to form a fluid and/or air tight seal or a substantially fluid and/or air tight seal.

The various embodiments of devices 100 of the present disclosure may comprise any number of suitable materials known the art useful with medical devices, such as various types of medical grade plastics. Furthermore, various embodiments of devices 100 may have any number of apertures 110 and/or notches 202 positioned thereon of various sizes and configurations so that, for example, one or more dialysis catheters or PICC lines may be positioned therein. In addition, the various embodiments of devices 100 may be suitable for single use (disposable) or for multiple uses.

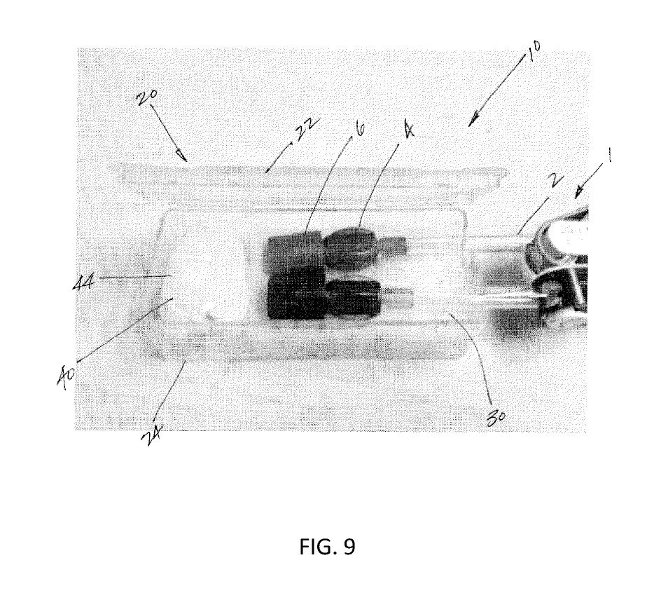

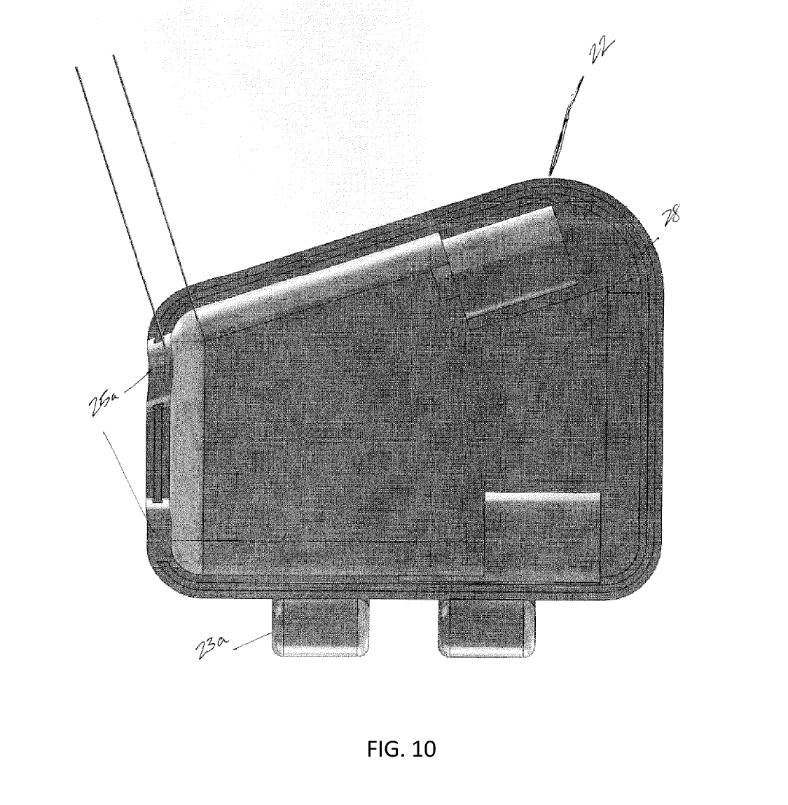

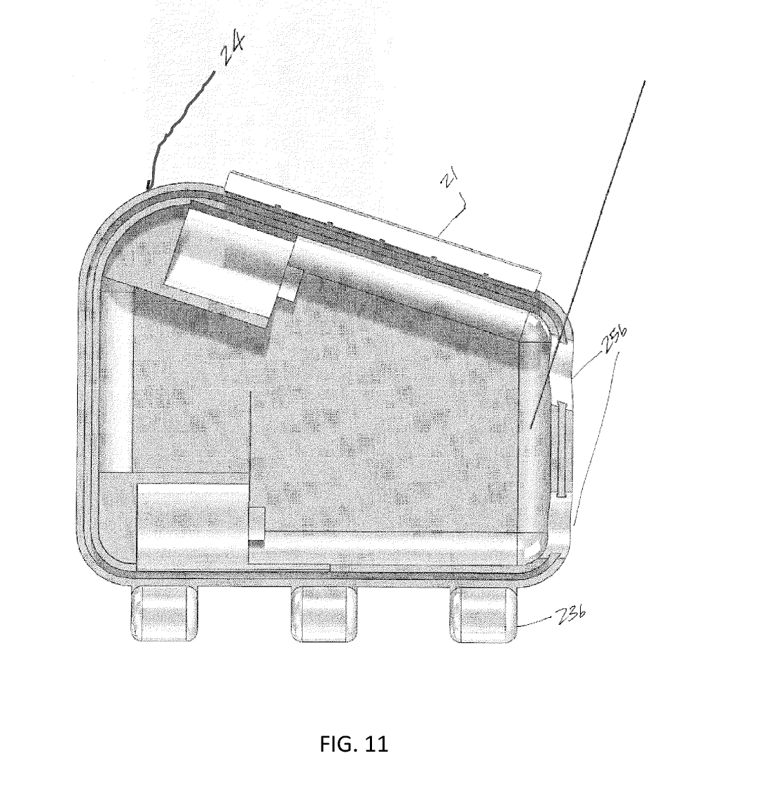

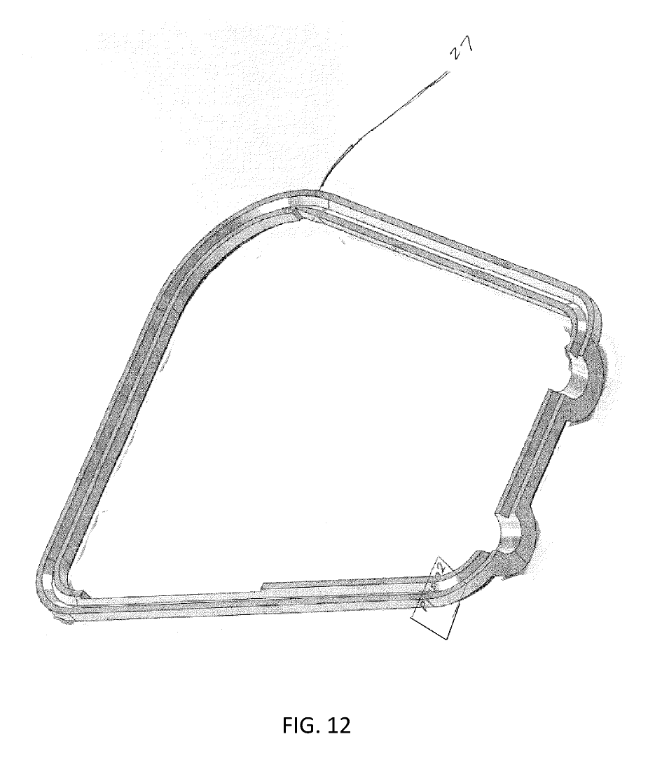

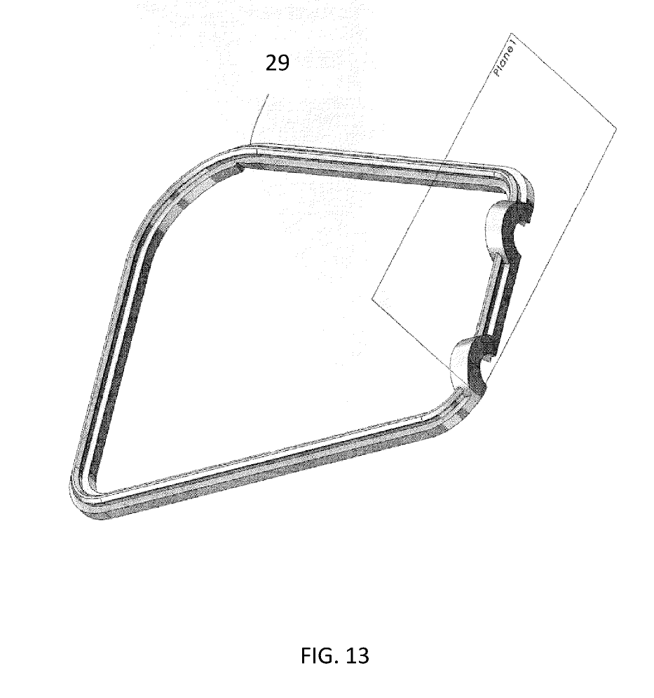

An exemplary embodiment of a device for maintaining an aseptic catheter environment of the present disclosure is shown in FIGS. 9-14. As shown in FIG. 9, an exemplary embodiment of device 1010 may include a box assembly 1020 having a sponge 1030 and an ampoule 1040 disposed therein. The box assembly 1020 may include a top housing 1022 movably attached by a hinge 1023 to a bottom housing 1024. The top housing 1022 may include a piercing member 1028 formed on an interior surface of the top housing 1022, the piercing member 1028 capable of puncturing the ampoule 1040. The top housing 1022 may further include one or more openings 1025a formed in a wall of the top housing 1022 to engage a catheter lead 1002 of a catheter 1001 as shown in FIG. 10. Likewise, the bottom housing 1024 may include one or more openings 1025b formed in a wall of the bottom housing 1024 to engage a catheter lead 1002 of a catheter 1001 as shown in FIG. 11. The hinge 1023 may include a top hinge portion 1023a formed integral or attached to the top housing 1022 and a bottom hinge portion 1023b formed integral or attached to the bottom housing 1024 as shown in FIGS. 10, 11, and 14.

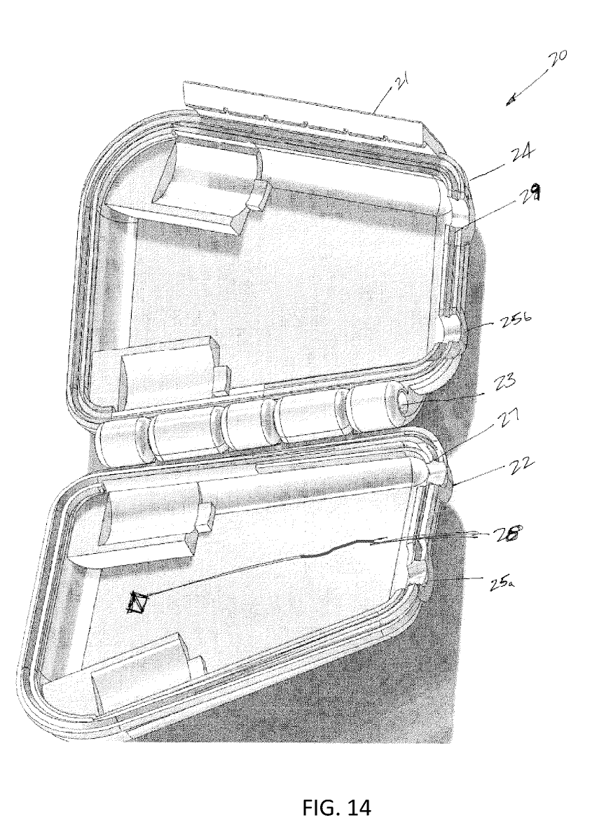

The box assembly 1020 may further include a top seal 1027 disposed within a channel formed adjacent the interior perimeter of the top housing 1022 and in the one or more openings 1025a. Similarly, the box assembly 1020 may further include a bottom seal 1029 disposed within a channel formed adjacent the interior perimeter of the bottom housing 1024 and in the one or more openings 1025b. FIGS. 9 and 14 show the box assembly 1020 in an opening configuration. The box assembly 1020 may also assume a closed configuration whereby the top housing 1022 is rotated about the huge 1023 such that the top housing 1022 seats against the bottom housing 1024 and whereby the top seal 1027 and bottom seal 1029 engage the top housing 1022 and bottom housing 1024 to seal the box assembly 1020. In the closed configuration, the openings 1025a and 1025b form one or more openings 1025. The bottom housing 1024 may include a latch 1021 formed integral or attached to the bottom housing 1024 and configured to engage and lock the top housing 1022 in position against the bottom housing 1024.

Referring to FIG. 9, in operation the catheter lead 1002 having a hub 1004 and a cap 1006 may be positioned within the bottom housing 1024 when the box assembly 1020 is in the open configuration. When the box assembly 1020 is closed into the closed configuration the catheter lead 1002 is sealed between the top seal 1027 and the bottom seal 1029 within the opening 1025. Once in the closed configuration, a force may be applied to the top housing 1022 to deflect the top housing 1022 and thereby driving the piercing member 1028 into and puncturing the ampoule 1040. The ampoule 1040 may be filled with a sterilizing fluid 1044, such as chlorohexidine. Once punctured, the ampoule 1040 may release the sterilizing fluid 1044 into the volume defined by the top housing 1022 and bottom housing 1024 in the closed configuration, thereby sterilizing the hub 1004, cap 1006, and lead 1002.

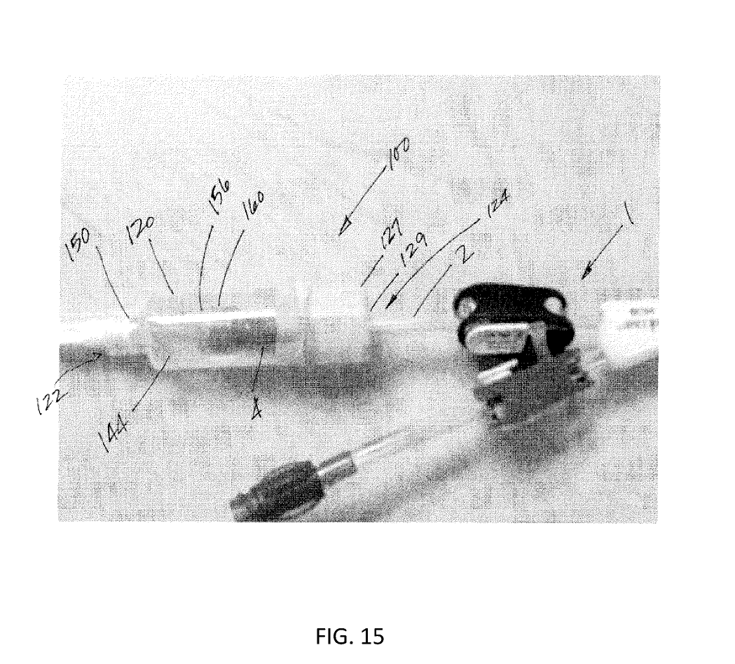

An alternative embodiment of a device for maintaining an aseptic catheter environment of the present disclosure is shown in FIGS. 15-17. As shown in FIG. 15, an exemplary embodiment of device 1100 may include a vial 1120 having a vial magnet 1150 disposed at a closed end 1122 and a seal cap 1127 disposed at an opposing insertion end 1124. The device 1100 may further include a sterilizing fluid 1144, such as chlorohexidine, disposed within the vial 1120 and a hub cap 1160 removably attached at the distal end of a hub 1004 of a catheter 1001. The hub cap 1160 may include a cap magnet 1156 attached thereto opposite an end that engages the hub 1004. The cap magnet 1156 may present a magnetic pole opposite that of the vial magnet 1150 such that the cap magnet 1156 and the vial magnet 1150 are magnetically attracted to one another.

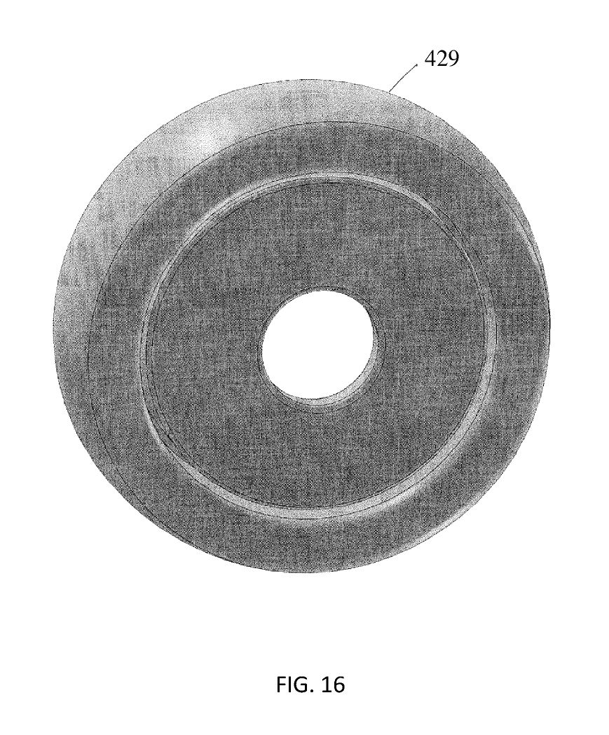

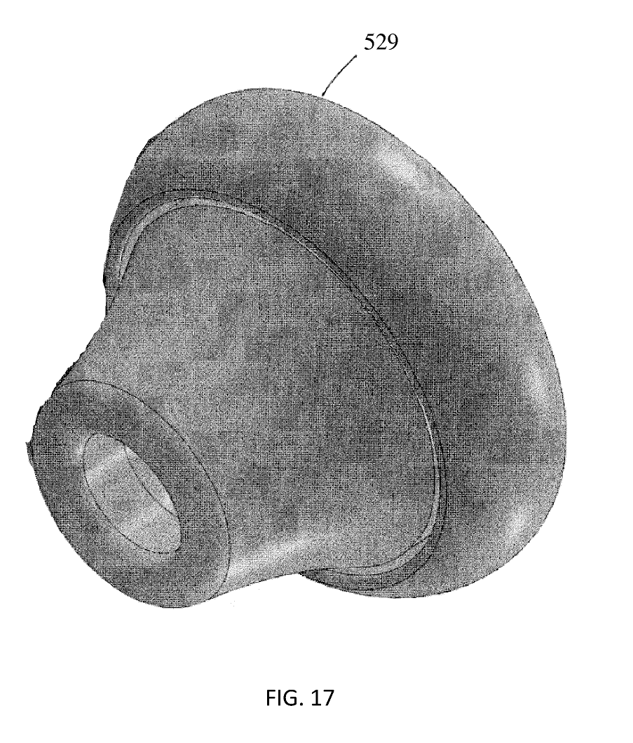

The seal cap 1127 may include a lead seal 1129 attached thereto, the lead seal 1129 formed with an aperture (not shown) capable of engaging and sealing a catheter lead 1002 disposed within the aperture. The seal cap 1127 and lead seal 1129 may be formed of resilient elastomeric materials. The seal cap 1127 and lead seal 1129 may further be formed to enable the hub 1004 and hub cap 1160 attached thereto to repeatedly pass through the aperture and maintain a reliable, water-tight seal. The lead seal 1129 may include the lead seal 1429 as shown in FIG. 16 and the lead seal 1520 as shown in FIG. 17.

In operation, the catheter lead 1002 having the hub 1004 and hub cap 1160 attached thereto at a distal end may be inserted through the aperture in the lead seal 1129 and into the sterilization fluid 1144 contained within the vial 1120, thereby sterilizing the hub 1004 and hub cap 1160. The catheter lead 1002 may be further advanced into the vial 1120 until the cap magnet 1156 and the vial magnet 1150 magnetically attach to one another, thereby preventing the catheter lead 1002 and hub 1004 from inadvertently disengaging the lead seal 1129 and vial 1120. Consequently, the hub 1004 and hub cap 1160 may be maintained within the sterile environment of the vial 1120 until a user applies sufficient force to disengage the cap magnet 1156 and the vial magnet 1150 from one another to withdraw the catheter lead 1002 from the vial 1120.

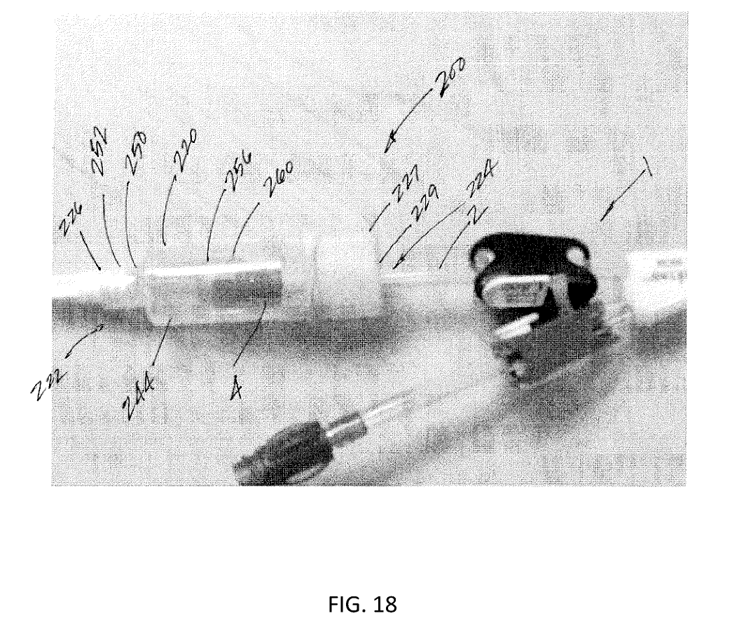

An alternative embodiment of a device for maintaining an aseptic catheter environment of the present disclosure is shown in FIG. 18. As shown in FIG. 18, an exemplary embodiment of device 1200 may include a vial 1220 having a vial magnet 1250 disposed at a refill end 1222 and a seal cap 1227 disposed at an opposing insertion end 1224. The device 1200 may further include a sterilizing fluid 1244, such as chlorohexidine, disposed within the vial 1220 and a hub cap 1260 removably attached at the distal end of a hub 1004 of a catheter 1001. The hub cap 1260 may include a cap magnet 1256 attached thereto opposite an end that engages the hub 1004. The cap magnet 1256 may present a magnetic pole opposite that of the vial magnet 1250 such that the cap magnet 1256 and the vial magnet 1250 are magnetically attracted to one another.

The vial magnet 1256 may be disposed upon a water-tight, one-way valve 1252 disposed within a valve opening 1226 formed in the refill end 1222 of the vial 1220. The valve 1252 may be formed to enable a fluid, such as the sterilizing fluid 1244, to be injected into the vial 1120 but to prevent fluid from leaving the vial 1120.

The seal cap 1227 may include a lead seal 1229 attached thereto, the lead seal 1229 formed with an aperture (not shown) capable of engaging and sealing a catheter lead 1002 disposed within the aperture. The seal cap 1227 and lead seal 1229 may be formed of resilient elastomeric materials. The seal cap 1227 and lead seal 1229 may further be formed to enable the hub 1004 and hub cap 1260 attached thereto to repeatedly pass through the aperture and maintain a reliable, water-tight seal. The lead seal 1229 may include the lead seal 1429 as shown in FIG. 16 and the lead seal 1520 as shown in FIG. 17.

In operation, the catheter lead 1002 having the hub 1004 and hub cap 1260 attached thereto at a distal end may be inserted through the aperture in the lead seal 1229 and into the sterilization fluid 1244 contained within the vial 1220, thereby sterilizing the hub 1004 and hub cap 1260. Alternatively, the sterilization fluid 1244 may be injected into the vial 1220 via the valve 1252 after the catheter lead 1002 has been inserted therein. The catheter lead 1002 may be further advanced into the vial 1220 until the cap magnet 1256 and the vial magnet 1250 magnetically attach to one another, thereby preventing the catheter lead 1002 and hub 1004 from inadvertently disengaging the lead seal 1229 and vial 1220. Consequently, the hub 1004 and hub cap 1260 may be maintained within the sterile environment of the vial 1220 until a user applies sufficient force to disengage the cap magnet 1256 and the vial magnet 1250 from one another to withdraw the catheter lead 1002 from the vial 1220.

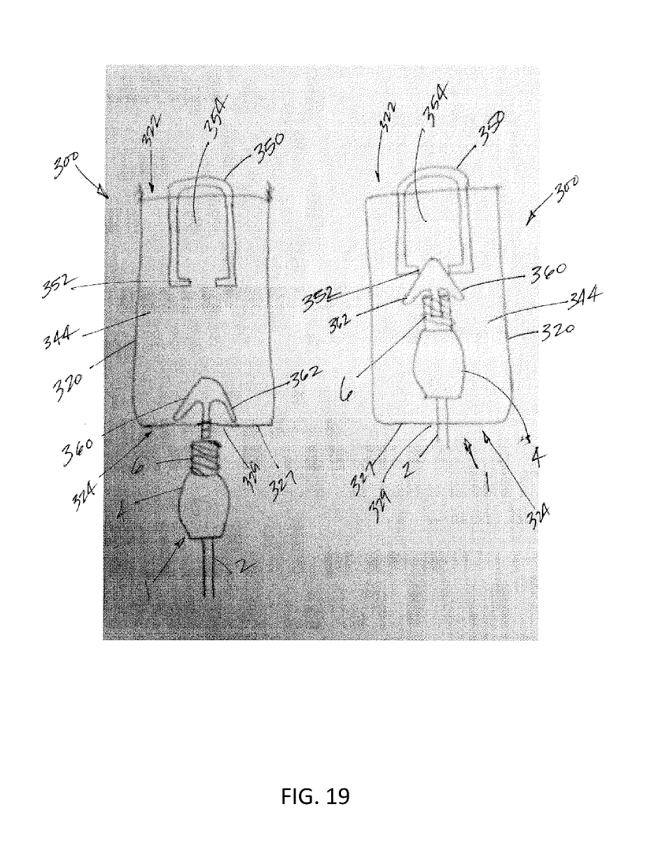

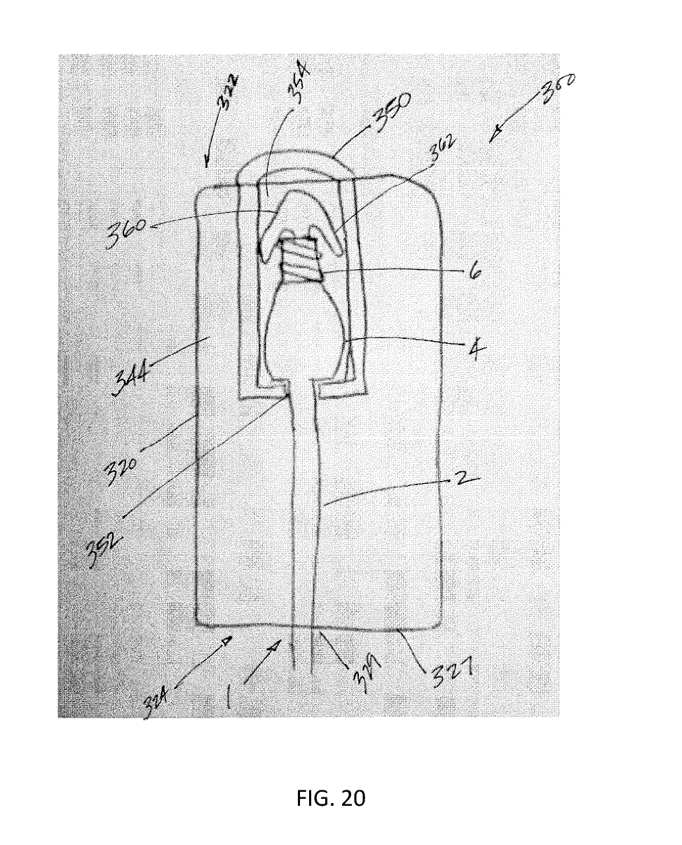

An alternative embodiment of a device for maintaining an aseptic catheter environment of the present disclosure is shown in FIGS. 19 and 20. As shown in FIG. 19, an exemplary embodiment of device 1300 may include a vial 1320 having a plug retainer 1350 disposed at a retention end 1322 and a seal cap 1327 disposed at an opposing insertion end 1324. The plug retainer 1350 may define a volume 1354 having a plug opening 1352 formed at a distal end within the vial 1320 and a proximal end outside the vial 1320. The device 1300 may further include a hub plug 1360 disposed adjacent the insertion end 1324 in an initial configuration. The device 1300 may further include a sterilizing fluid 1344, such as chlorohexidine, disposed within the vial 1320.

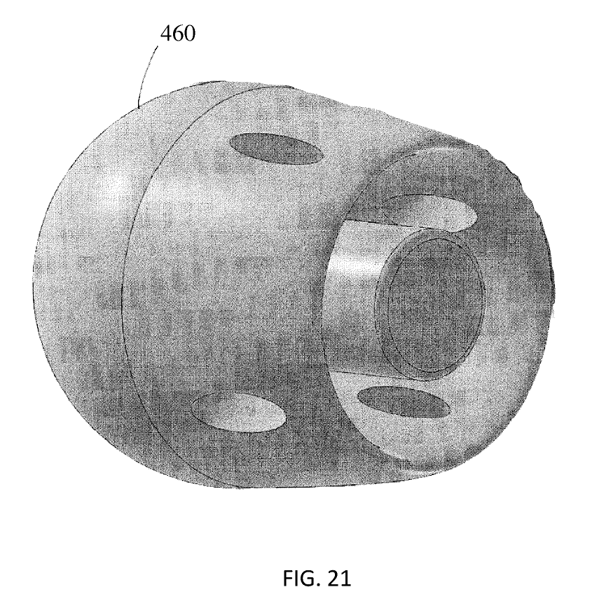

The seal cap 1327 may include a lead seal 1329 attached thereto, the lead seal 1329 formed with an aperture (not shown) capable of engaging and sealing a catheter lead 1002 disposed within the aperture. The seal cap 1327 and lead seal 1329 may be formed of resilient elastomeric materials. The seal cap 1327 and lead seal 1329 may further be formed to engage and retain at least a portion of the hub plug 1360 within the aperture in the initial configuration. Further, the seal cap 1327 and lead seal 1329 may be formed to enable the hub 1004 to repeatedly pass through the aperture and maintain a reliable, water-tight seal. The lead seal 1329 may include the lead seal 1429 as shown in FIG. 16 and the lead seal 1520 as shown in FIG. 17. The hub plug 1360 may include the hub plug 1460 as shown in FIG. 21.

The hub plug 1360 may be formed to removably attach to and seal the distal end of the hub 1004 of the catheter 1001. Further, the hub plug 1360 may include retention features 1362 that prevent the hub plug 1360 from being withdrawn from the vial 1320 via the aperture in the lead seal 1329.

In operation, the catheter lead 1002 having the hub 1004 attached thereto at a distal end may be inserted through the aperture in the lead seal 1329, thereby engaging the hub plug 1360 such that the hub plug 1360 seals access to the interior of hub 1004 and catheter lead 1002. The catheter lead 1002 may be further advanced into the vial 1320 and the sterilization fluid 1344 contained therein, thereby sterilizing the hub 1004 and a plurality of hub threads 1006, which may be formed in the exterior surface of the hub. The catheter lead 1002 may be further advanced through the plug opening 1352 of the plug retainer 1350 and into the volume 1354 until the proximal end of the hub 1004 passes through the plug opening 1352 as shown in FIG. 20. FIG. 20 depicts a locked configuration of the catheter lead 1002 within the device 1300. The locked configuration prevents the catheter lead 1002 and hub 1004 from inadvertently disengaging the lead seal 1329 and vial 1320. Consequently, the hub 1004 and hub threads 1006 may be maintained within the sterile environment of the vial 1320 until a user intentional disengages the plug retainer 1350 and withdraws the hub 1004 from the hub plug 1360, thereby returning the device 1300 to the initial configuration.

The plug retainer 1350 may be disengaged by any means that enables hub 1004 and hub plug 1360 to pass back through the plug opening 1352 without disengaging the hub plug 1360 from the hub 1004. In at least one embodiment, the walls of the plug retainer 1350 may flex so as to enlarge the plug opening 1352 when a force is applied to the proximal end of the plug retainer 1350.

In at least one embodiment of the present disclosure, an antiseptic catheter cap may include a body having volume defined by an enclosed wall having a proximal end, a distal end, and a side wall extending there between as shown in FIG. 9. The catheter cap is capable of accepting, retaining, and providing an antiseptic environment for a catheter port.

The catheter cap may further include a membrane disposed at the proximal end, the membrane having an orifice formed therethrough. The membrane may be an elastic material capable of deforming significantly without rupturing. The membrane is capable of wrapping around and sealing the catheter volume after the port is pushed through it. Although as shown in FIG. 9, the membrane will be attached to the device. The catheter cap may further include a port cap disposed between the body and membrane, which is attached to the wall of the device by very elastic medical tubing/material. The material has holes in it so the chlorhexidine solution can get through and clean as much of the catheter port as possible. The port cap stretches like a rubberband as the port is pushed through the port cap and into the device. The stretching enables a tight seal on the catheter port and keeps the cap on the port without covering threads of the catheter port. The port would stay plugged until the catheter is pulled out of the device. Dispersion of the chlorhexidine solution would take place by pushing the catheter port through the membrane, where it would be capped automatically, then it would be pushed into the back of the device to rupture a chlorihexidine ampoule within the body volume at the distal end of the device. The ampoule is represented as a sphere in FIG. 9. The body of the catheter cap may be a clear plastic to allow for visual confirmation of the dispersal of the solution as well as confirmation of a proper seal for the port. The catheter cap has various advantages over conventional caps, including easy of manufacture on a large scale.

In at least one embodiment according to the present disclosure, the catheter cap may include a winged capsule having hinges on the side and a perimeter seal disposed between the wings as shown in the FIGS.

While various embodiments of devices and methods for maintaining an aseptic catheter environment have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure. Further, where the embodiments illustrated in the drawings, and specific language will be used to describe the same, refer a body or a patient's body, it will be understood that the subject body may be a human body or an animal body, as the use of catheters in vivo is known in both human and veterinary medicine.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

What is claimed is:

1. A device for maintaining an aseptic catheter environment, the device comprising:
a first housing portion and a second housing portion, the first housing portion and the second housing portion coupled to one another by way of at least one hinge, the first housing portion and the second housing portion configured to engage one another when in a closed configuration and to define an interior space when in the closed configuration;
at least one clip coupled to the first housing portion or the second housing portion, the at least one clip operable to secure the first housing portion to the second housing;
a fluid sac containing an antiseptic fluid, the fluid sac positioned upon at least one of the first housing portion and the second housing portion; and
at least one of an aperture or notch defined within at least one of the first housing portion and the second housing portion, the at least one of an aperture or notch is sized and shaped to allow at least a portion of a catheter to be positioned therethrough.

2. A method of using a device for maintaining an aseptic catheter environment, the method comprising the steps of:
placing a distal end of a bodily catheter within at least one of an aperture or a notch of a device for maintaining an aseptic catheter environment; and
activating the device to release an antiseptic fluid within the device.

3. The method of claim 2, wherein the device has an open position and a closed position, and the method further includes the step of moving the device to the closed position prior to activating the device to release an antiseptic fluid within the device.

4. The method of claim 2, wherein the step of activating the device is performed by applying pressure to the device to cause the antiseptic fluid within the device to be released.

5. The method of claim 2, wherein the step of activating the device is performed following the step of placing the distal end of the bodily catheter within at least one of the aperture or the notch of the device.

6. A device for maintaining an aseptic catheter environment, the device comprising:
a vial having a plug retainer disposed at a retention end and a seal cap disposed at an opposing insertion end, the vial being at least partially filled with a sterilizing fluid, wherein the plug retainer defines a volume having a plug opening formed at a distal end within the vial and a proximal end outside the vial; and
a lead seal attached to the seal cap, the lead seal comprising an aperture formed to enable a hub and catheter lead to pass therethrough and further formed to engage a hub plug disposed with the aperture adjacent the insertion end in an initial configuration.
